(19) United States
(12) Patent Application Publication
(10) Pub. No.: US 2015/0044738 A1
(43) Pub. Date: Feb. 12, 2015

(54) PRODUCTION OF EICOSAPENTAENOIC AND/OR ARACHIDONIC ACID IN MIXOTROPHIC MODE BY EUGLENA

(71) Applicant: FERMENTALG, Libourne (FR)

(72) Inventors: Khadidja Romari, Clermont-ferrand (FR); Pierre Calleja, Bordeaux (FR)

(73) Assignee: FERMENTALG, Libourne (FR)

(21) Appl. No.: 14/385,507

(22) PCT Filed: Mar. 15, 2013
(86) PCT No.: PCT/FR2013/050540

§ 371 Date: Sep. 16, 2014

(30) Foreign Application Priority Data
Mar. 16, 2012 (FR) 1252376

(51) Int. Cl. C12P 7/64; C12R 1/89

(52) U.S. Cl. CPC C12P 7/6427; C12R 1/89
USPC 435/134; 435/257.4; 435/252

(57) ABSTRACT

New strains of microalgae belonging to the Euglena genus, allow production of lipids, in particular of EPA and/or ARA, in mixotrophic mode, and a method for selecting and culturing such strains, using a variable and/or discontinuous light source, in particular a flashing light.

The invention relates to a method of culture in mixotrophic mode, in particular in the presence of discontinuous and/or variable illumination with light, of a microalga of the genus Euglena, in particular of the species Euglena gracilis. The method makes it possible to obtain a high yield of biomass and enrichment of the thus cultured microalgae in lipids and more particularly in eicosapentaenoic acid (EPA) and/or arachidonic acid (ARA). The method thus makes it possible to select strains of Euglena gracilis with mixotrophic character, and having a high yield of lipids and more particularly of polyunsaturated fatty acids. The invention also relates to a novel strain of microalga belonging to the species Euglena gracilis, particularly suitable for the production of fatty acids. This novel strain of Euglena gracilis is useful for producing EPA (eicosapentaenoic acid) and arachidonic acid (ARA) in mixotrophic mode.

### Preamble

Microalgae are autotrophic photosynthetic microorganisms, i.e. they have the ability to grow autonomously by photosynthesis.

Microalgae develop both in the marine aquatic media and in fresh or brackish waters, as well as in various land habitats.

Most species of the microalgae found in fresh water or in the oceans are generally autotrophic, i.e. they can only grow by photosynthesis. For these species, the presence in their environment of carbon-containing substrates or organic matter is not favourable to them and does not improve their growth.

However, a certain number of species of microalgae, of very varied families and origins, are found to be not strictly autotrophic. Thus, some of them, said to be heterotrophic, are capable of developing in the total absence of light, by fermentation, i.e. by using organic matter.

Other species of microalgae, for which photosynthesis remains essential for their development, are capable of benefiting both from photosynthesis and from organic matter present in their environment. These intermediate species, said to be mixotrophic, can be cultured in the presence of both light and organic matter.

This particularity of so-called “mixotrophic” algae seems to be related to their metabolism, which allows them to carry out photosynthesis and fermentation simultaneously. Both types of metabolism co-exist with a positive overall effect on the growth of the algae [Yang, C. et al. (2000) Biochemical Engineering Journal 6: 87-102].

Microalgae are currently the subject of numerous industrial projects since some species are capable of accumulating or secreting major quantities of lipids, in particular polyunsaturated fatty acids.

Among these polyunsaturated fatty acids, certain highly unsaturated fatty acids from the omega-3 series (PUFA-ω3), in particular eicosapentaenoic acid (EPA or C20:5 ω3) and docosahexaenoic acid (DHA or C22:6 ω3), and from the omega-6 series (PUFA-ω6), in particular arachidonic acid (AA or ARA or also eicosatetraenoic acid 020:4 ω6) have a recognized nutritional importance, and have strong potential in terms of therapeutic applications.

Fish oils, from the fishing industry, are currently the main commercial source of these types of fatty acids. However, while these oils find new applications (food supplement in aquaculture, incorporation into margarines), marine halieutic resources are becoming scarce because of intensive fishing activity.

New sources of these fatty acids such as EPA and ARA, therefore, have to be sought in order to meet, in the future, the increasing demand for these types of polyunsaturated fatty acids.

In addition to their capability of synthesizing fatty acids de novo, microalgae provide several advantages compared to fish oils: they may be cultured in vitro under controlled conditions, which allows production of a biomass of a relatively constant biochemical composition, and, in addition, unlike fish oils, they do not have an unpleasant smell and their lipids contain little or no cholesterol.

Finally, the lipids produced by microalgae have a simpler fatty acid profile than that of fish oils, which limits the steps for separating the fatty acids of interest.

At present, the classification of algae is still widely based on morphological criteria and on the character of the photosynthetic pigments that their cells contain. Consequently, it is not very indicative of the autotrophic, heterotrophic or mixotrophic character of the algal species, whereas the latter cover a very great diversity of species and forms [Dubinsky et al. (2010), Hydrobiologia, 639: 153-171]. The taxonomic classification of eukaryotic algae contains 14 phyla. Large variations exist among the species of the different classes making up these phyla that produce fatty acids, as regards the polyunsaturated fatty acid content of the microalgae. Moreover, the relative proportions of lipids, in particular of EPA and of ARA in the lipid profiles, vary according to the species and the culture conditions.

The main microalgae of interest, producing EPA and ARA, are marine species. However, among the hundreds of thousands of species of marine microalgae, only a small number have a high content of both of these two fatty acids and sufficient capacity to be cultured in vitro. The species of interest are mainly Bacillariophytes (or diatoms) from marine phytoplankton. They are generally characterized by an active production of EPA.

Although they are rich in α-linolenic acid (C18:3 ω3), fresh water microalgae generally do not contain EPA [Pencreac'h et al. (2004) Les microalgues marines: source alternative d'EPA et de DHA, Lipides, 11(2):118-222].

In contrast, the production of ARA is quite rare for marine algae. Such microalgae include but are not limited to, Euglenophytes (for example, Euglena), Rhodophytes (for example, Porphyridium) and Chlorophytes (for example, Parietochloris).

Several strains of fresh water microalgae are capable of synthesizing and accumulating arachidonic acid. Among others, the triglycerides of the Chlorophyte Parietochloris incisa, an alga isolated from Mont Tateyama in Japan, are naturally rich in arachidonic acid. ARA is mainly present in the form of triglycerides which constitute the dominant class of lipids [Bigogno C. et al. (2002); Lipid and fatty acid composition of the green oleaginous alga Parietochloris incisa, the richest plant source of arachidonic acid, Phytochemistry, 60(5): 497-503].

For implementing the production of fatty acids by microalgae on an industrial scale, several factors must be taken into account. For example, cultures may be carried out under autotrophic, mixotrophic or heterotrophic conditions depending on the strain, the temperature, the lighting conditions and the size of the fermenters. For example, cultures may also be carried out in 1 L containers, in a laboratory, in photo-bioreactors, and in 100,000 litres containers or in open ponds (several hectares). However, the costs of energy and other resources such as manpower and the ease of monitoring the culture must be taken into account for developing ideal culture conditions.

In any case, it is desirable that the microalgae are cultured under optimum conditions for increasing the yield of the fatty acid(s) to be produced. Thus, it is preferable to have a yield that is as high as possible (for example biomass above 30 g/l of dry matter, and more than 15% of fatty acids by weight relative to the dry matter).

International patent application WO 03/079810 discloses cultures of Parietochloris incisa for the production of ARA intended for animal feedstuffs. These cultures were carried out under autotrophic conditions with a yield of ARA of at least 10% ARA by weight with respect to the weight of these cells.

Japanese patent applications JP9252764 and JP60087798 also describe strains of Monodus subterraneus cultured in autotrophic mode capable of accumulating a quantity of EPA ranging up to 3.8% of their dry weight. These strains were cultured under laboratory conditions, i.e. in inorganic culture media, in flasks or bioreactors having a low-volume capacity with a continuous light source.

From the standpoint of industrial exploitation, such a method of culture proves to be unsuitable. In fact, to be profitable, it must be possible for biomass production to be carried out in large, closed photo-bioreactors. Now, such a method of culture is difficult to implement in autotrophic mode, because when the density of the cells increases in the culture medium, it becomes more and more difficult for the cells to capture the light originating from outside the reactor. It is therefore necessary to actively stir the culture medium, which requires substantial energy expenditure.

It would be desirable to be able to obtain greater yields of EPA and ARA for a more efficient and more cost-effective industrial production.

Euglena is a common genus of flagellate protists and forms part of the family of the Euglenoids which do not have a stiff wall, which gives their mobility a characteristic form while in motion, called euglenoid movement. Euglena can lose their chloroplasts and produce heterotrophic depigmented individuals that are not very different from the flagellate protozoa. Euglena have a stigma, an orange-coloured mark that fulfills the role of photoreceptor and enables the micro-organism to navigate. The flagellae are permanently present in Euglena. The metabolism of Euglena is polyvalent. In the presence of light, they are autotrophic, and in the absence of light or after the loss of their chloroplast, they become heterotrophic and are in particular capable of metabolizing lactates [Lechevalier, Hubert A. (1977); Fungi, Algae, Protozoa, and Viruses, CRC Handbook of Microbiology, 2nd edition, Vol. II, p. 874, Floride, Laskin, Allen I.].

In the scientific article “Biomass production in mixotrophic culture of Euglena gracilis under acidic condition and its growth energetics”, Biotechnology Letters Vol. 23, No. 15, 1223-1228, it was demonstrated that the biomass yield obtained with Euglena gracilis cultured under mixotrophic conditions (with continuous light) is from 15 to 19% greater than the yield obtained with a culture under heterotrophic conditions. Moreover, high levels of chlorophyl of 39.4 mg per litre and of carotenoids of 13.8 mg per litre have been obtained in the mixotrophic cultures.

In the scientific article by Hayashi M et al.: “Enriching Euglena with unsaturated fatty acids”, BIOSCIENCE BIOTECHNOLOGY BIOCHEMISTRY, vol. 57, no 2, 1 Jan. 1993, p. 352-353, the enrichment in EPA of the microalgae of the genus Euglena gracilis is described. The microalgae are cultured under heterotrophic conditions with an addition of EPA or DHA from 0 to 1% to the culture medium. In order to obtain microalgae with a level of 75.6% of EPA with respect to their total lipids, it was necessary to add 0.5% of EPA to the culture medium, which represents a step that is expensive and not cost-effective for industrial production.

Thus, it was after numerous experiments under unusual lighting conditions and with the addition of various substrates that the applicant succeeded in isolating microalgal strains of the species Euglena gracilis that can be cultured in mixotrophic mode, allowing, under the conditions of the present invention, a high-yield production of polyunsaturated fatty acids, in particular EPA and ARA.

One strain (FCC 540), representing novel strains of Euglena gracilis thus isolated and selected, was deposited at the CCAP (Culture Collection of Algae and Protozoa, Scottish Association for Marine Science, Dunstaffnage Marine Laboratory, Oban, Argyll PA37 1QA, Scotland, United Kingdom) according to the provisions of the Treaty of Budapest, under the accession number CCAP 1224/49.

The method of culture and selection consisted more particularly of culturing the microalgae under mixotrophic conditions, in the presence of variable and/or discontinuous illumination, notably in the form of flashes, with a range of specific variations of light intensity and frequency.

The frequent alternation of illuminated phases and phases of darkness or of lower light intensity, generally perceived as stressful for microalgae, surprisingly made it possible to obtain a high production of biomass, of lipids and more particularly of polyunsaturated fatty acids from the strains of Euglena gracilis. The application of such strains according to the invention opens the perspective of industrial production of polyunsaturated fatty acids, in particular of EPA and ARA, in fermenters benefiting from a reduced light supply, and should therefore make possible energy savings compared with autotrophic modes of culture.

The different aspects and advantages of the invention are detailed below.

### DETAILED DESCRIPTION

The present invention therefore relates to a method of culture of microalgae of the genus Euglena, in particular of the species Euglena gracilis, in mixotrophic mode, under conditions of illumination that is discontinuous and/or variable over time. The illumination has variations in intensity, the amplitude of which is generally comprised between 5 μmol.m⁻².s⁻¹ and 1,000 μmol.m⁻².s⁻¹, preferably between 30 and 400 μmol.m⁻².s⁻¹.

These variations can generally take place between 2 and 3,600 times per hour, preferably between 2 and 200 times per hour. These culture conditions make it possible to supply a defined quantity of light. This light supply may comprise phases of discontinuous and/or variable illumination, with variations in intensity that may have identical or different amplitudes. The illumination may in particular be in the form of flashes.

The advantage of this method is to increase the yield of biomass obtained from the culture. The other is to enrich the thus cultured microalgae in polyunsaturated fatty acids, more particularly in eicosapentaenoic acid (EPA) and/or arachidonic acid (ARA). This method can also be used for selecting strains of the genus Euglena, in particular of the species Euglena gracilis, with a mixotrophic character and having a high yield of polyunsaturated fatty acids, in particular of EPA (eicosapentaenoic acid) and/or of ARA (arachidonic acid).

The culture of this microalga in mixotrophic mode is preferably carried out in the presence of 5 mM to 1 M, preferably from 50 mM to 800 mM, more preferably from 70 mM to 600 mM, and even more preferably from 100 mM to 500 mM of an organic carbon-containing substrate. The substrate is supplied continually during the culture, so as to allow the cells to accumulate a significant concentration of lipids. Additional substrate is added to the culture medium during the culture so as to maintain a constant concentration. This organic carbon-containing substrate preferentially comprises, in pure form or as a mixture: glucose, derivatives of cellulose, lactate, starch, lactose, saccharose, acetate and/or glycerol.

The organic carbon-containing substrate contained in the culture medium may consist in complex molecules or in a mixture of substrates. The products resulting from the biotransformation of starch, for example starting from maize, wheat or potato, notably, starch hydrolysates, which consist of small sized molecules, for example, form organic carbon-containing substrates which may be used for mixotrophic culture of the microalgae according to the invention.

This method is more particularly intended for the use of novel strains of microalgae of the genus Euglena (Phylum: Euglenoza, Order: Euglenales, Family: Euglenaceae) [ITIS Catalogue of Life, 2010] selected for their mixotrophic character, notably for their capability to be cultured with a light supply greater than 10 μE, in a mineral medium, for example the Euglena medium [Andersen, R. A.; Jacobson, D. M. & Sexton, J. P. (1991)—Provasoli-Guillard Center for Culture of Marine Phytoplankton, Catalogue of Strains. 98pp. West Boothbay Harbor, Me., USA], to which an organic carbon-containing substrate is added. Preferably, the organic carbon-containing substrate comprises glucose and/or lactate, at a concentration that is equivalent to or greater than 5 mM.

These novel strains of Euglena, more particularly Euglena gracilis, may be isolated and selected by the method of selection and culture according to the invention described hereafter.

A representative strain of the Euglena gracilis strains according to the invention is the FCC 540 strain isolated by the applicant and deposited at the CCAP, under the accession number CCAP 1224/49. Such strains are capable of producing significant quantities of biomass as well as lipids, and more particularly of EPA and ARA, when they are cultured in mixotrophic mode with a variable or discontinuous light supply, according to the invention.

According to ongoing taxonomic analyses, the strain CCAP 1224/49 belongs to the species Euglena gracilis. The invention relates to any strain of the species Euglena gracilis, capable of growing under mixotrophic culture conditions such as described in the present application, and capable of producing fatty acids, such as EPA and ARA. The invention also relates to any strain of microalga of the genus Euglena, capable of growing under mixotrophic culture conditions such as described in the present application, and capable of producing fatty acids, such as EPA and ARA.

The isolated strains of Euglena gracilis according to the invention make it possible to produce, under mixotrophic conditions, significant quantities of biomass as well as lipids rich in EPA and/or ARA, said EPA and/or ARA being capable of representing more than 10%, more than 25%, or more than 40% of the total lipids contained in the microalgae.

In the present invention, the biomass obtained with the FCC 540 strain, isolated by the applicant, from a culture under mixotrophic conditions in the presence of variable and/or discontinuous illumination, notably in the form of flashes, is from 10 to 60%, more generally from 20 to 50%, greater than that of a culture with the same strain carried out in heterotrophic mode. By heterotrophic mode, it is meant identical culture conditions, with the exception of the absence of illumination.

The present invention thus relates to a method for culturing microalgae of the genus Euglena, in particular of the species Euglena gracilis in mixotrophic mode, in the presence of an illumination that is variable and/or discontinuous over time, for example, in the form of flashes, notably with a view to producing polyunsaturated fatty acids, such as EPA and ARA.

The present invention thus relates to a method for selecting strains of the genus Euglena, in particular of the species Euglena gracilis, with a mixotrophic character, and having a high yield of polyunsaturated fatty acids such as EPA and ARA, in the presence of an illumination that is variable and/or discontinuous over time.

It appeared that variable and/or discontinuous illumination of the cultures, in particular when used in a culture in mixotrophic mode, had a favourable impact on the development of the algae and made it possible to increase the productivity of the latter, notably as far as their lipid production is concerned.

Without being bound to theory, the inventor believes that a discontinuous and/or variable light supply to the microalgae has the effect of causing a “stress” favourable to the growth and to the synthesis of lipids. This phenomenon may be partly explained by the fact that, in nature, microalgae tend to accumulate lipid reserves to withstand the constraints of their environment.

By discontinuous illumination, it is meant illumination punctuated by periods of darkness. The periods of darkness may be more than a quarter of the time, preferably, half of the time or more, during which the algae are cultured.

According to a preferred aspect of the invention, the illumination is discontinuous and, more preferentially, in the form of flashes. A flash, within the meaning of the invention, is illumination with light of short duration, i.e. of less than 30 minutes. The duration of the flash may be less than 15 minutes, preferably less than 5 minutes or even more preferentially less than 1 minute. According to certain embodiments of the invention, the duration of the flash may be less than a second. For example, the duration of the flash may be 1/10 of a second, or 2/10 of a second, or 3/10 of a second, or 4/10 of a second, or 5/10 of a second, or 6/10 of a second, or 7/10 of a second, or 8/10 of a second, or 9/10 of a second. The illumination with light, or the flash, generally lasts longer than 15 seconds. The duration of the flash is generally comprised between 5 seconds and 10 minutes, preferably between 10 seconds and 2 minutes, more preferentially between 20 seconds and 1 minute.

In general, the number of flashes is comprised between about 2 and 3,600 per hour. It may be, for example, comprised between 100 and 3,600 flashes per hour. It may also be comprised between 120 and 3,000, or between 400 and 2,500, or between 600 and 2,000, or between 800 and 1,500 flashes per hour. It may also be comprised between 2 and 200, preferentially between 10 and 150, more preferentially between 15 and 100, and even more preferentially between 20 and 50 per hour. The flashes may be emitted at regular or irregular time intervals. In the case of emission at regular intervals, the number of flashes per hour then corresponds to a frequency (F) having a time period (T), it being considered that F=1/T. This time period may be comprised between 1 second and 30 minutes, or between 1 second and 36 seconds, or between 1.2 second and 30 seconds, or between 1.44 second and 9 seconds, or between 1.8 second and 6 seconds, or between 2.4 seconds and 4.5 seconds. This frequency may also be comprised between 18 seconds and 30 minutes, preferentially between 24 seconds and 6 minutes, more preferentially between 36 seconds and 4 minutes, and even more preferentially between 72 seconds and 3 minutes. The number of flashes per hour is selected as a function of the intensity and duration of the flashes (see below). In general, the intensity of the light supplied in the form of flashes is between 5 and 1,000 μmol.m⁻².s⁻¹, preferably between 5 and 500 μmol.m⁻².s⁻¹, or 50 and 400 μmol.m⁻².s⁻¹, and more preferentially between 150 and 300 μmol.m⁻².s⁻¹. By definition, 1 μmol.m-².s⁻¹ corresponds to 1μm⁻².s⁻¹ (Einstein), a unit which is frequently used in the literature.

According to a particular embodiment of the invention, the intensity of the light is comprised between 50 and 200 μmol.m⁻².s⁻¹, the frequency of the flashes is comprised between 10 seconds and 60 minutes for a flash duration comprised between 1 second and 1 minute.

According to another embodiment of the invention, the illumination may be variable, which means that the illumination is not interrupted by phases of darkness, but instead the light intensity varies over time. This variation of the light intensity is regular and may be periodic or cyclic.

According to the invention, light may also be supplied combining phases of continuous and discontinuous illumination.

According to the invention, regardless of the illumination conditions, the light intensity supplied to the algae in culture, expressed in micromoles of photons per second per square metre (μmol.m⁻².s⁻¹), varies at least once in any one hour. The amplitude of this variation of light intensity is generally between 5 and 1,000, or between 50 and 800, or between 100 and 600 μmol.m⁻².s⁻¹. The intensity of the light can also vary between 5 and 400 μmol.m⁻².s⁻¹. Preferably, the amplitude of the variation of light intensity is between 70 and 300 μmol.m⁻².s⁻¹ and more preferentially between 100 and 200 μmol.m⁻².s⁻¹.

Said light intensity may attain successively, under conditions of variable illumination, for example, the values 50 μmol.m⁻².s-¹ and 100 μmol.m⁻².s⁻¹, or 5 and 400 μmol.m⁻².s⁻¹, or 50 and 800 μmol.m⁻².s⁻¹ several times every hour. Said light intensity can attain successively, preferably, the values 50 and 200 μmol.m⁻².s⁻¹. Alternatively, under conditions of discontinuous illumination, said light intensity can attain successively, several times per hour, for example, the values 0 and 50 μmol.m⁻².s⁻¹, the values 0 and 100 μmol.m⁻².s⁻¹ or still more preferentially the values 0 and 200 μmol.m⁻².s⁻¹. It can also attain successively, several times per hour, for example, the values 0 and 300 μmol.m⁻².s⁻¹, the values 0 and 600 μmol.m⁻².s⁻¹, the values 0 and 800 μmol.m⁻².s⁻¹ or also the values 0 and 1,000 μmol.m⁻².s⁻¹.

According to an embodiment of the invention, regardless of the illumination conditions, the intensity of the light supplied to the culture varies as a function of the cell density. The denser the culture becomes, the more intense the light may be. The cell density is the number of cells per ml and it is measured by the techniques known to one skilled in the art.

At the initial stage of the culture when the cell density is between approximately 10⁵ and 5×10⁵ cells per ml, the light intensity can be comprised between 5 and 15 μmol.m⁻².s⁻¹, preferably between 5 and 10 μmol.m⁻².s⁻¹. When the culture reaches a density between 10⁶ and 10⁷ cells per ml, the light intensity can be increased to between 15 and 200 μmol.m⁻².s⁻¹, for example, preferably, between 20 and 50 μmol.m⁻².s⁻¹. When the culture, at the final stage, reaches a density between 10⁷ and 10⁸ cells per ml, the light intensity can be increased to between 50 and 400 μmol.m².s⁻¹ for example, preferably, between 50 and 150 μmol.m².s⁻¹.

According to certain embodiments, for example when the duration of the flashes is for example less than a minute, or less than a second, the intensity of the light may be higher than the values stated above. At the initial stage of the culture, when the cell density is between approximately 10⁵ and 5×10⁵ cells per ml, the light intensity can be between 5 and 200 μmol.m⁻².s⁻¹, preferably between 5 and 100 μmol.m⁻².s⁻¹. When the culture reaches a density between 10⁶ and 10⁷ cells per ml, the light intensity can be increased to between 30 and 500 μmol.m⁻².s⁻¹, for example, preferably, between 50 and 400 μmol.m⁻².s⁻¹. When the culture, at the final stage, reaches a density between 10⁷ and 10⁸ cells per ml, the light intensity can be increased to between 100 and 1,000 μmol.m⁻².s⁻¹ for example, preferably between 200 and 500 μmol.m⁻².s⁻¹.

According to an embodiment of the invention, the quantity of light supplied to the culture per hour remains between certain values. It is comprised between about 2,000 and 600,000, preferably between 2,000 and 300,000 μmol.m⁻². It can be comprised between 4,000 and 200,000 μmol.m⁻², per hour.

According to an embodiment of the invention, the culture is illuminated with 30 flashes per hour, each flash having a duration of 30 seconds and an intensity of 10 μmol.m⁻².s⁻¹. The latter gives a total supply of light per hour of 9,000 μmol.m⁻². According to another embodiment of the invention, the culture is illuminated with 20 flashes per hour, each flash having a duration of 30 seconds and an intensity of 20 μmol.m⁻².s⁻¹. The latter gives a total supply of light per hour of 12,000 μmol.m⁻². According to another embodiment of the invention, the culture is illuminated with 45 flashes per hour, each flash having a duration of 15 seconds and an intensity of 5 μmol.m⁻².s⁻¹, which gives a total supply of light per hour of 3,375 μmol.m⁻². According to another embodiment of the invention, the culture is illuminated with 120 flashes per hour, each flash having a duration of 10 seconds and an intensity of 200 μmol.m⁻².s⁻¹, which gives a total supply of light per hour of 240,000 μmol.m⁻².

As described for the light intensity above, and according to an embodiment of the invention, the quantity of light supplied to the culture per hour may vary as a function of the cell density. At the initial stage of the culture, when the cell density is 10⁵ and 5×10⁵ cells per ml, the total supply of light per hour is generally comprised between approximately 1,500 and 8,000, preferably 1,500 and 6,000 μmol.m⁻², yet more preferably between 2,000 and 5,000 μmol.m⁻². When the culture reaches a density between 10⁶ and 10⁷ cells per ml, the total supply of light per hour can be increased until between 6,000 and 67,000 μmol.m⁻², preferably between 6,000 and 50,000, and yet more preferably between 12,000 and 45,000 μmol.m⁻², for example. In the final stage of culture, at a cell density between 10⁷ and 10⁸ cells per ml, the total supply of light per hour may be increased to between 45,000 and 300,000, for example preferably between 45,000 and 200,000 μmol.m⁻², and for example, more preferably, between 50,000 and 150,000 μmol.m⁻².

According to an embodiment of the invention, at the initial stage of the culture (at a cell density between 10⁵ and 5×10⁵ cells per ml), the culture is illuminated with 30 flashes per hour, each flash having a duration of 30 seconds and an intensity between 5 and 10 μmol.m⁻².s⁻¹, which gives a total supply of light per hour of 2,250 μmol.m⁻² to 4,500 μmol.m⁻². Then, at the intermediate stage (at a cell density between 10⁶ and 10⁷ cells per ml), the culture is illuminated with 30 flashes per hour, each flash having a duration of 30 seconds and an intensity between 15 and 50 μmol.m⁻².s⁻¹, which gives a total supply of light per hour of 13,500 to 45,000 μmol.m⁻². Then, at the final stage of the culture (at a cell density between 10⁷ and 10⁸ cells per ml), the culture is illuminated with 30 flashes per hour, each flash having a duration of 30 seconds and an intensity between 50 and 150 μmol.m⁻².s⁻¹, which gives a total supply of light per hour of 45,000 to 135,000 μmol.m⁻².

According to an embodiment of the invention, for example, when the duration of the flashes is for example of less than a minute, or less than a second, at the initial stage of the culture (at a cell density between 10⁵ and 5×10⁵ cells per ml), the culture is illuminated with 30 flashes per hour, each flash having a duration of 10 seconds and an intensity between 50 and 100 μmol.m⁻².s⁻¹, which gives a total supply of light per hour of 15,000 μmol.m⁻² to 30,000 μmol.m⁻². Then at the intermediate stage (at a cell density between 10⁶ and 10⁷ cells per ml), the culture is illuminated with 50 flashes per hour, each flash having a duration of 10 seconds and an intensity between 200 and 300 μmol.m⁻².s⁻¹, which gives a total supply of light per hour of 100,000 to 150,000 μmol.m⁻². Then, at the final stage of the culture (at a cell density between 10⁷ and 10⁸ cells per ml), the culture is illuminated with 120 flashes per hour, each flash having a duration of 10 seconds and an intensity between 350 and 450 μmol.m⁻².s⁻¹, which gives a total supply of light per hour of 420,000 to 540,000 μmol.m⁻².

The light supply to the cultures may be obtained by lamps distributed around the external wall of the fermenters. A clock triggers these lamps for defined illumination times. The fermenters are preferentially located in an enclosure, shielded from daylight, whose ambient temperature may be controlled.

As the applicant could ascertain, the fact that the thus selected strains have good growth capabilities in mixotrophic mode, in the presence of discontinuous and/or variable light, predisposes said strains to a higher production of polyunsaturated fatty acids, notably EPA and ARA.

The method of culture according to the invention thus allows selection of strains of the genus Euglena, in particular the species Euglena gracilis, with mixotrophic character, similar to that isolated by the applicant and deposited at the CCAP under the accession number CCAP 1224/49, and having a high yield of polyunsaturated fatty acids. This method of culture is characterized in that it comprises the following steps:

a) culture, in mixotrophic mode, of one or more strains of the genus Euglena, under conditions of illumination that is discontinuous and/or variable over time, the illumination having variations in intensity, the amplitude of which is comprised between 5 μmol.m⁻².s⁻¹ and 1,000, preferably between 5 and 400 μmol.m⁻².s⁻¹, these variations taking place between 2 and 3,600, preferably 5-400 times per hour,

b) a step of maintaining said culture over several generations, in the presence of an organic carbon-containing substrate in the culture medium, and optionally

c) a step of recovery of the thus cultured microalgae.

By “step of recovery”, it is meant more particularly the isolation of the strain or strains for which the number of cells increased the most during said generations. For carrying out selection of the strains, different strains of the genus Euglena, in particular of the species Euglena gracilis, may be cultured, in parallel, on microplates in one and the same enclosure, with precise monitoring of the conditions and evolution of the different cultures. It is, thus, easy to determine the response of the various strains to discontinuous and/or variable illumination and, if applicable, to the addition of one or more organic carbon-containing substrates to the culture medium.

The strains that respond favourably to the discontinuous and/or variable illumination and to the organic carbon-containing substrates, generally provide a better yield for the production of lipids in terms of quality (polyunsaturated fatty acids more abundant in the lipid profile) and in terms of quantity (the lipids contain a higher proportion of EPA and ARA).

The microalgae may be selected in a fermenter from a heterogeneous population, and from which one aims to select the variants favoured by the manner of selection according to the invention, combining discontinuous and/or variable light, having a specific range of light intensity and a specific frequency, with mixotrophic culture conditions. In this case, culture is carried out by maintaining the microalgae in culture over many generations, and then an isolation of the components that have become predominant in the culture medium, is performed at the end of culture.

The method of culture according to the invention also makes it possible to produce lipids.

In this case, the method according to the invention further comprises the following steps:

d) a step of recovery of the lipids from the microalgae, and optionally

e) the extraction of eicosapentaenoic acid (EPA) and/or arachidonic acid (ARA) from the recovered lipids.

The method of culture according to the invention may also be applied to any species of the genus Euglena gracilis, capable of growing under the mixotrophic conditions according to the invention, and capable of producing EPA and/or ARA.

The method of culture according to the invention makes it possible to optimize the production of the biomass obtained from the culture. It also makes it possible to enrich the thus cultured microalgae in polyunsaturated fatty acids, more particularly in eicosapentaenoic acid (EPA) and/or arachidonic acid (ARA).

Therefore, the invention is also directed to optimizing the production of biomass, as well as the production of lipids, in particular of fatty acids, through the culture of microalgae of the genus Euglena with mixotrophic character, preferably cultured or selected according to the methods mentioned above, then the recovery of the thus cultured microalgae in order to extract the lipids therefrom, in particular EPA and/or ARA. The strains of the species Euglena gracilis are especially concerned.

The methods for selectively extracting the lipids, including EPA and ARA, are known to one skilled in the art and are, for example, described by [Bligh, E. G. and Dyer, O. K. (1959); A rapid method of total lipid extraction and purification, Can. J. Biochem. Physiol., 37:911-917].

The invention also relates to the microalgae of the genus Euglena gracilis, capable of being obtained according to the method of the invention as previously described. These microalgae are enriched in polyunsaturated fatty acids. The total lipids of such microalgae generally comprise more than 15% or 20%, often more than 30% and sometimes even more than 40% of EPA and/or ARA with respect to the total percentage of lipids.

### EXAMPLE 1

The cultures of Euglena gracilis FCC 540 are carried out in 2 L usable capacity fermenters (bioreactors) with dedicated automatic controllers with computerized supervision. The pH of the system is adjusted by adding base (a 1N sodium hydroxide solution) and/or acid (a 1N sulphuric acid solution). The culture temperature is set to 22° C. Stirring is achieved using 2 stirring rotors mounted on the shaft according to the Rushton configuration: three-blade impellers with downward pumping. The stirring speed and the aeration flow are regulated to a minimum=100 rpm and to a maximum=250 rpm and Qmin=0.5 vvm/Qmax=2 vvm respectively. The bioreactor is equipped with an external lighting system surrounding the transparent tank. The intensity as well as the light cycles are controlled by a dedicated automatic device with computerized supervision.

The reactors are inoculated with a preculture prepared on a stirring table (140 rpm) in a controlled-temperature enclosure (22° C.) and illuminated between 80 and 100 μE. Pre-cultures and cultures in the bioreactors are carried out in the Euglena medium [Starr, R. C. & Zeikus, J. A. (1993)—UTEX—The Culture Collection of Algae at the University of Texas at Austin. J. Phycol. Suppl. 29]. The organic carbon-containing substrate used for the mixotrophic culture in a bioreactor is glucose at concentrations of between 100 mM and 150 mM.

### Monitoring of the Cultures:

The total biomass concentration is monitored by measuring the dry mass (filtration on a Whatman GFC filter, and then oven drying in vacuo, at 65° C. and −0.8 bars, for a minimum of 24 h before weighing).

Regarding the quantification of the total number of lipids, 10⁸ cells/mL were extracted. Methods for extracting lipids are known to one skilled in the art.

### Illumination:

The culture is illuminated with 30 flashes per hour, each flash having a duration of 30 seconds and an intensity of 80 μmol.m⁻².s⁻¹.

The light supply to the cultures in the bioreactor was obtained by LED (light-emitting diode) lamps distributed around the external wall of the fermenter. A clock triggers these LEDs for illumination times or pulses.

Results (n=3):

| | Dry | Total lipids | | |
|---|---|---|---|---|
| | mass | (% of dry | ARA (% of | EPA (% of |
| | (g/L) | mass) | total lipids) | total lipids) |
| Mixotrophy | 25.6 +/− 1.2 | 17.1 +/− 0.7 | 15.6 +/− 0.3 | 14 +/− 0.9 |
| with flashes | | | | |
| Heterotrophy | 17.5 +/− 1 | 18.7 +/− 0.2 | 16.1 +/− 0.4 | 14.3 +/− 1.1 |

1. Method comprising the following step:
a) culture, in mixotrophic mode, of one or more strains of the genus Euglena under conditions of illumination that is discontinuous and/or variable over time, the illumination having variations in intensity, the amplitude of which is comprised between 5 and 1,000, these variations taking place between 2 and 3,600 times per hour.

2. Method according to claim 1, characterized in that the illumination has variations in intensity, the amplitude of which is comprised between 5 μmol.m⁻².s⁻¹ and 400 μmol.m⁻², these variations taking place between 2 and 200 times per hour.

3. Method according to claim 1, characterized in that the microalga is from the species Euglena gracilis.

4. Method according to claim 1, characterized in that the culture is carried out in the presence of an organic carbon-containing substrate at a concentration from 5 mM to 1 M, preferably from 50 mM to 800 mM, more preferentially from 70 mM to 600 mM, and even more preferentially from 100 mM to 500 mM, the organic carbon-containing substrate being selected from starch, lactate, lactose, saccharose, acetate, glycerol, glucose and cellulose derivatives and a mixture of these molecules.

5. Method according to claim 4, characterized in that said organic carbon-containing substrate present in the culture medium comprises at least 5 mM of glucose.

6. Method according to claim 5, characterized in that the amplitude of the variations in intensity is comprised between 30 and 400 μmol.m⁻².s⁻¹, preferably between 70 and 300 μmol.m⁻².s⁻¹, and more preferentially between 100 and 200 μmol.m⁻².s⁻¹.

7. Method according to claim 1, characterized in that the light supply is in the form of flashes.

8. Method according to claim 7, characterized in that a flash has a duration comprised between 1/10 second and 10 minutes, preferably between 5 seconds and 10 minutes, more preferably between 10 seconds and 2 minutes, even more preferentially between 20 seconds and 1 minute.

9. Method according to claim 7, characterized in that the number of flashes is between 5 and 3,600, preferably between 10 and 150, more preferentially between 15 and 100, and even more preferentially between 20 and 50 times per hour.

10. Method according to claim 1, characterized in that the total light supply per hour in micromoles of photons is between 2,000 to 600,000 μmol.m⁻², preferably between 2,000 to 200,000 μmol.m⁻².

11. Method according to claim 1, characterized in that it further comprises the following steps:
b) a step of maintaining said culture over several generations in the presence of an organic carbon-containing substrate in the culture medium, and optionally
c) a step of recovery of the thus cultured microalgae, and optionally
d) a step of recovery of the lipids from the microalgae, and optionally
e) the extraction of eicosapentaenoic acid (EPA) and/or arachidonic acid (ARA) from the recovered lipids.

12. Microalga of the genus Euglena gracilis capable of being obtained by the method of claim 1.

13. Microalga of the genus Euglena, according to claim 12, characterized in that its total lipids comprise more than 10%, preferably more than 25%, and more preferentially, more than 40% of EPA and/or ARA with respect to the total percentage of lipids.

14. Method according to claim 2, characterized in that the microalga is from the species Euglena gracilis.
